(19) United States
(12) Patent Application Publication
(10) Pub. No.: US 2024/0042438 A1
(43) Pub. Date: Feb. 8, 2024

(54) DEVICES AND METHODS FOR SPECTROSCOPY OF BIOMATERIALS AND LIVE CELLS

(71) Applicant: The Regents of the University of California, Oakland, CA (US)

(72) Inventors: Hoi-Ying N. Holman, Oakland, CA (US); Wujun Zhao, Waunakee, WI (US); Sankar Raju Narayanasamy, Oakland, CA (US)

(21) Appl. No.: 18/489,182

(22) Filed: Oct. 18, 2023

Related U.S. Application Data
(63) Continuation of application No. PCT/US2022/025689, filed on Apr. 21, 2022.
(60) Provisional application No. 63/178,769, filed on Apr. 23, 2021.

(51) Int. Cl. B01L 3/00

(52) U.S. Cl. CPC B01L 3/502746; B01L 2200/12; B01L 2300/12; B01L 2200/027; B01L 2300/0681; G01N 2021/3595

(57) ABSTRACT

This disclosure provides systems, methods, and apparatus related to infrared spectroscopy. In one aspect, a device includes a first assembly, a porous membrane, and a second assembly. The first assembly defines a fluid distributor. The porous membrane overlies the fluid distributor and forms a surface of the fluid distributor. The second assembly is disposed on the first assembly. The second assembly defines a plurality of capillary arrays surrounding a window in the second assembly that exposes the porous membrane. Capillaries of each of the capillary arrays have openings on edges of the window of the second assembly and are operable to direct moisture across a surface of the porous membrane.

### RELATED APPLICATIONS

This application claims priority to PCT Application No. PCT/US2022/025689, filed Apr. 21, 2022, and to U.S. Provisional Patent Application No. 63/178,769, filed Apr. 23, 2021, both of which are herein incorporated by reference. This application is related to U.S. Pat. No. 9,927,352, granted Mar. 27, 2018, and to U.S. Pat. No. 11,054,367, granted Jul. 6, 2021, both of which are herein incorporated by reference.

### STATEMENT OF GOVERNMENT SUPPORT

This invention was made with government support under Contract No. DE-ACO2-O5CH11231 awarded by the U.S. Department of Energy and Award No. DE-SC0019228 awarded by the U.S. Department of Energy Office of Biological and Environmental Research (BER) Bioimaging Science Program. The government has certain rights in this invention.

### BACKGROUND

The circular bioeconomy—wherein renewable feedstocks are sustainably utilized to offset energy and valuable materials conventionally produced with petroleum—is a key component to achieving worldwide carbon neutrality. Cellulose from lignocellulosic biomass is the most abundant biopolymer on the planet. On the one hand, complete depolymerization of cellulose yields soluble glucose, an excellent feedstock for conversion to fuels or chemicals. On the other, cellulose can be processed into nanofibrils and nanocrystals with superior mechanical properties for use with advanced biomaterials. Efficient and environmentally friendly processes to achieve either saccharification or nanomaterial production are best achieved by enzyme-mediated reactions.

Controlling enzyme catalyzed reaction kinetics, however, has long been a challenge. Cellulose hydrolysis is characterized by rapidly declining reaction rates despite the remaining abundance of cellulose in the reaction, which has been postulated to be limited by the accessibility of cellulose to cellulases. The interaction of cellulase enzymes with cellulose is an interfacial reaction on structurally heterogeneous cellulosic materials. An understanding of spatial and temporally resolved changes of cellulose structural properties during enzyme hydrolysis reaction is needed for controlling bulk reaction kinetics. There have been intense research efforts aimed at increasing the understanding of the currently working mechanism of enzymatic cellulose hydrolysis involving binding of a cellulase to a cellulose surface, complexation with a single cellulose molecule within the catalytic domain of the enzyme, and processive hydrolytic cleavage of glycosidic bonds in the cellulose chain. Cellulases bind to cellulose in a variety of modes, where “productively bound” enzymes are those that form active complexes with a molecule of cellulose and the fibril surface, and “non-productively bound” enzymes are bound to cellulose but are either not complexed or are in a complexed but inactive state.

Fourier transform infrared (FTIR) spectromicroscopy is one of the most sensitive physical techniques for studying spatially resolved changes in biomolecular structure and composition. FTIR has been widely used to characterize cellulosic materials in pretreatment design and nanomaterial production. This physical approach involves collecting FTIR spectra on vacuum- or oven-dried samples and quantifying ratios of absorbance peaks (i.e., crystallinity index) in the fingerprint region and the hydride-OH region assigned to hydrogen bonding patterns characteristics of cellulose. FTIR spectra of cellulose on dried post-treated cellulosic samples has demonstrated changes in cellulose crystallinity, crystalline morphology, and intrafibril hydrogen bonding attributable to enzymatic action. Recent studies show that FTIR requires controlling the sample environment (e.g., surface tension, concentration, humidity, temperature); for example, variance in surface tension spatially localizes and concentrates cellulosic materials on the FTIR plate during sample drying, which causes localized strong infrared peak intensities and negatively affects sample-to-sample reproducibility. In many cases, both pre-treated and post-treated cellulose samples are subjected to different sample preparation methods (e.g., sample separation, sample buffer exchange, sample wash), contributing to variations in the FTIR spectral signals.

For example, one research group reported evidence of decrystallization and preference for Iα or Iβ crystalline morphology of Thermobifida fusca Cel5A, Cel6B, and Cel9A cellulases. They quantified peak ratios in the FTIR spectra of enzyme treated and dried samples. A follow-up study by another research group with a sample preparation method of multiple cycles of centrifugation, buffer-exchange, and washing of digestion residues reported no conclusive impact of the enzyme action at different stage of hydrolysis on cellulose crystallinity.

These studies highlight the challenge of analyzing post-treated and dried samples and emphasize the need for methods that enable in situ time- and spatially-resolved studies of cellulose undergoing hydrolysis by cellulases. While sample drying improves the ease of data acquisition by removing strongly infrared-absorbant water, the most representative cellulose structure requires the presence of moisture. These FTIR studies of cellulose structure were unlikely to capture the hydration-dependent cellulose crystallinity critical for studying depolymerization catalysis, such as effects of hydrogen bond network.

### SUMMARY

Described herein is an open-channel microfluidic membrane device. This device can be used for long-term enzymatic hydrolysis and analysis of cellulose ordering during its de-polymerization in humid or hydrated environments. Embodiments of the device include features relative to standard microfluidic membrane devices, including hydraulic resistors to modulate the passive flow rates in the microfluidic, a device layout capable of automatically coupling lateral and vertical capillary imbibition sources to maintain humidity, capillary design features to achieve the automatic coupling, and vertical interconnects to dispense reagents or collect products on-demand, which allow for real-time detection of spatiotemporal changes of hydrogen bond network in cellulose using standard hyperspectral microscopy. The device allows for autonomous control and multiplexed measurement and analysis with a significant reduction in human intervention, with no tedious and conflicting post treatment sample preparation, representing a direct recording of the depolymerization event as it is unfolding in situ and in real time.

The device was used in acquiring data for structural and compositional analysis of the crystalline cellulose fibrils during bioenergy or nanomaterial production. These fibrils were purified from the green algae Cladophora aegagropila undergoing enzymatic hydrolysis during biofuel production with the cellobiohydrolase enzyme (Cel7A). Under favorable low temperature conditions (representing a four-fold increase relative to hydrolysis control) at 37° C. and a relative humidity slightly below saturation, a decline in peak intensity of the C—O—C glycosidic bond in the infrared fingerprint and shifts of peaks from C—O stretches at C6 and C3 to higher frequencies were observed. The appearance of new peaks of D(CO) of the C—O—C glycosidic linkage and increases in the intensities of the peaks also were observed. As the process transpired, from the hydrogen-bonded OH stretching vibrations localized changes in the crystalline structure were observed—with over 50% of the areas exhibiting decreases in molecular ordering (crystallinity), suggesting active complexation and hydrolysis activity by cellulases at these regions. Other regions, however, exhibited increased crystallinity, suggesting that cellobiohydrolase preferentially removes ‘less ordered’ cellulose and increases the overall crystallinity. Less often, areas with fluctuations in crystallinity were observed. These effects also were observed for higher temperatures and can address unresolved conflicts in the field of feedstock for conversion to fuels and chemicals. The capillary-based microfluidics spectroscopic imaging device described herein is a new tool for applications in renewable energy, biotechnology, material science, environmental science, and other fields.

One innovative aspect of the subject matter described in this disclosure can be implemented in device including a first assembly, a porous membrane, and a second assembly. The first assembly defines a first fluid resistor, a fluid distributor, and a second fluid resistor. The first fluid resistor, the fluid distributor, and the second fluid resistor are connected in series. The first fluid resistor is in fluid communication with the fluid distributor. The fluid distributor is in fluid communication with the second fluid resistor. The porous membrane overlies the fluid distributor. The porous membrane forms a surface of the fluid distributor. The second assembly is disposed on or over the first assembly. The second assembly defines a third fluid resistor and a plurality of capillary arrays surrounding a window in the second assembly that exposes the porous membrane. The third fluid resistor and the plurality of capillary arrays are in fluid communication. The device further defines a working fluid inlet in fluid communication with the first fluid resistor, a working fluid outlet in fluid communication with the second fluid resistor, a water inlet in fluid communication with third fluid resistor, and a water outlet in fluid communication with the plurality of capillary arrays. Capillaries of each of the capillary arrays have openings on edges of the window of the second assembly and are operable to direct moisture across a surface of the porous membrane.

In some implementations, the porous membrane is a porous membrane selected from the group silicon nitride, silicon dioxide, silicon/silicon dioxide, and graphene. In some implementations, the porous membrane is a silicon nitride porous membrane. In some implementations, the porous membrane is about 50 nanometers to 500 microns thick. In some implementations, the porous membrane has dimensions of about 3 millimeters to 5 millimeters by about 3 millimeters to 5 millimeters. In some implementations, the porous membrane defines a plurality of pores, with each pore of the plurality of pores having a size of about 100 nanometers to 2 microns. In some implementations, a layer of a metal is disposed on the surface of the porous membrane.

In some implementations, a number of capillaries in each capillary array is about 100 to 1000. In some implementations, the plurality of capillary arrays consists of four capillary arrays, the window has a square shape, and each of the capillary arrays has openings on one of the sides of the window. In some implementations, the window has a square shape, and the window has dimensions of about 1 millimeter to 4 millimeters by about 1 millimeter to 4 millimeters. In some implementations, the capillaries of each of the capillary arrays have dimensions of about 1 micron to 35 microns by about 1 micron to 35 microns. In some implementations, the height of the capillary is less than the width of the capillary. In some implementations, a channel defining the third fluid resistor has dimensions of about 1 micron to 35 microns by about 1 micron to 35 microns.

In some implementations, channels defining the first fluid resistor, the fluid distributor, and the second fluid resistor have dimensions of about 1 micron to 35 microns by about 1 micron to 35 microns.

In some implementations, the first assembly and the second assembly each comprise a polymer from the group polydimethyl siloxane (PDMS), polyamide (PA), polycarbonate (PC), polyester, polyethylene (PE), poly(ethylene terephthalate) (PET), poly(ethylene terephthalate glycol) (PETG), poly(methylmethacrylate) (PMMA), polystyrene (PS), poly(tetrafluoroethylene (PTFE), polyurethane (PU), poly(vinyl chloride) (PVC), cellulose acetate (C), and cyclic olefin copolymer (COC).

In some implementations, the first assembly is about 1 millimeter to 3.5 millimeters thick. In some implementations, the second assembly is about 0.4 millimeters to 1.2 millimeters thick.

In some implementations, the device further comprises an adhesive membrane disposed between the first assembly and the second assembly. In some implementations, the first assembly is bonded to the second assembly via plasma bonding.

Details of one or more embodiments of the subject matter described in this specification are set forth in the accompanying drawings and the description below. Other features, aspects, and advantages will become apparent from the description, the drawings, and the claims. Note that the relative dimensions of the following figures may not be drawn to scale.

### BRIEF DESCRIPTION OF THE DRAWINGS

FIGS. 1A-1G show examples of schematic illustrations of a device. FIG. 1H shows an example of a flow diagram illustrating a method of operating a device.

FIGS. 2A and 2B show sFTIR spectra of dried cellulose and cellulose in sodium acetate buffer in the ‘hydrogen bonding’ region (3150-3500 cm⁻¹) featuring—OH vibration peaks of cellulose (FIG. 2A) and in the fingerprint region (900-1300 cm⁻¹) dominated by C—O vibrational peaks (FIG. 2B).

FIG. 3A shows time-resolved FTIR spectra of cellulose undergoing evaporative dehydration in a humidity-controlled microfluidics device set at 25% rh at 37° C. Absorption peaks at −2100 cm−1 and 1650 cm⁻¹ associated with free water are marked with *; scale-bar indicates 0.5 absorbance units. FIG. 3B shows time course absorbances of peaks centered at 1040 cm−1 and 1650 cm⁻¹.

FIG. 4A shows uncorrected spectra of enzyme in buffer (Enzyme), dried cellulose (Dried), and cellulose in buffer with enzymes at 0 hours, 1 hour, and 12 hours at 38° C. FIG. 4B shows background subtracted spectra (900-1200 cm⁻¹) recorded at 10 minute intervals from time 0 to 12 hours.

FIG. 5 shows a ‘heat map’ of the evolution of the peak intensity of the glycosidic bond peak at −1160 cm⁻¹ over 14 hours. Each square shows the region where the FTIR spectra were obtained at the reaction time indicated.

### DETAILED DESCRIPTION

Reference will now be made in detail to some specific examples of the invention including the best modes contemplated by the inventors for carrying out the invention. Examples of these specific embodiments are illustrated in the accompanying drawings. While the invention is described in conjunction with these specific embodiments, it will be understood that it is not intended to limit the invention to the described embodiments. On the contrary, it is intended to cover alternatives, modifications, and equivalents as may be included within the spirit and scope of the invention as defined by the appended claims.

In the following description, numerous specific details are set forth in order to provide a thorough understanding of the present invention. Particular example embodiments of the present invention may be implemented without some or all of these specific details. In other instances, well known process operations have not been described in detail in order not to unnecessarily obscure the present invention.

Various techniques and mechanisms of the present invention will sometimes be described in singular form for clarity. However, it should be noted that some embodiments include multiple iterations of a technique or multiple instantiations of a mechanism unless noted otherwise.

The terms “about” or “approximate” and the like are synonymous and are used to indicate that the value modified by the term has an understood range associated with it, where the range can be ±20%, ±15%, ±10%, ±5%, or ±1%. The terms “substantially” and the like are used to indicate that a value is close to a targeted value, where close can mean, for example, the value is within 80% of the targeted value, within 85% of the targeted value, within 90% of the targeted value, within 95% of the targeted value, or within 99% of the targeted value.

Water absorbs infrared light strongly. It has long been recognized that microfluidics, a technology that exploits fluid flow in micron-dimension channels, represents a potential boon for controlling sample environment involving real-time FTIR spectromicroscopy of in situ biochemical processes. Specifically, these channels can maintain a 10 micron to 20 micron thick laminar-flow-through system, which minimizes both the imaging volume in liquid and the signal interference from geometry-induced fringing. Channels have also developed for tracking the locations and concentrations of molecules in live cellular systems over time.

Recently, similar microchannels have been developed to investigate kinetics of enzymatic treatment of plant biomass in bioenergy production. These studies are interesting, but the absorption even from a 10 micron thick water layer still saturates the infrared signal in the hydride-OH spectral region, obscuring chemical bands associated with the intermolecular and intramolecular hydrogen-bonding interactions. Microfluidics have never before been used to maintain an infrared-compatible water thin film for use as a discovery tool to study the spectral features in the hydride OH region. This would enable, for example, the identification of molecular conditions that could result in enhanced formation of active complexes in which a molecule of cellulose is bound by cellulases during bioenergy or commercial cellulosic material productions.

A promising alternative to microchannels for integration of FTIR methods for cellulose depolymerization analysis is an open-channel microfluidic membrane (OMM) device for the study of live adherent mammalian cell systems. Composed of a coated porous membrane between a feeding channel and an enclosed viewing chamber, an OMM device allows cells to be maintained on the upper membrane surface in a layer of fluid while media is replenished from the feeding channel below. In OMM devices using a micropore membrane, a liquid layer is produced on the membrane through capillary flow and is known to flood the cells, obscuring the infrared signals. In nanopore OMM microfluidics, a thin liquid layer is produced on the nanopore membrane through processes similar to those controlling adsorption and capillary condensation-induced imbibition in nanoporous media. The nanopore OMM possesses unique features that are not present in channel-based or micromembrane-based microfluidic devices. For example, in nanopore OMM, the rate of liquid (e.g. water, buffer) uptake through the nanopores and the condensation on the membrane surface can be manipulated by a number of parameter, such as the pore size, the relative humidity in the chamber, and the chemical potentials at the inlet boundary. Micropore OMM has been used previously for a number of applications related to mammalian cell-cultures and analysis.

Described herein is an OMM-based device for FTIR spectromicroscopy. Also described is the use of such a device to observe the spatial and time-resolved changes in molecular ordering of crystalline cellulose undergoing enzymatic hydrolysis at an atmospheric relative humidity below saturation. Some researchers have reported that the most representative cellulose structure studied by FTIR is in the presence of moisture to capture hydrogen bonding effects. Other researchers reported that the maximum degree of ordering of the cellulose structure occurs at an unsaturated atmospheric relative humidity below 85%.

The devices and methods described herein, which rely on autonomously controlling the relative humidity over the micropore or nanopore membrane, were used to track: (1) during enzymatic hydrolysis at micrometer scale the evolution of the cellulose features C-0 bonds at five of the six carbons of the glucose residues that contribute to infrared absorption peaks in the ‘carbohydrate region’; and (2) the features of hydroxyl groups in the three of the six carbons forming intermolecular and intramolecular hydrogen bonds that contribute to infrared absorption peaks in the OH-hydride region of the FTIR spectra. Time-dependent patterns of spatial changes in the prominent absorption peaks corresponding to C—O stretches at the sixth (C6) and third carbon (C3) of the glucose residues, and to the glycosidic bond C—O—C that links ring-shaped sugar molecules to other molecules in the cellulose, were documented. Moreover, spatiotemporal changes in the absorption peaks arising from the pair of O5 . . . HO3 and O2H . . . O6 intramolecular hydrogen bonds that brace the glycosidic bond, and to peaks corresponding to the OH . . . O type hydrogen bonds that are interconnected to form flat sheets with the CH . . . O hydrogen bond were recorded. Importantly, the discovery of a new spectral feature called glycosidic bond index (GBI), demonstrating a new insight in the hydrogen bonds analysis, structure determination, and the likely morphology characterization is reported.

By better understanding the molecular cellulose-cellulase interactions that drive these localized changes over time, results from this imaging approach will provide key insights for improving future design of more efficient cellulase or of next generation energy plants that are easier to be hydrolyzed enzymatically.

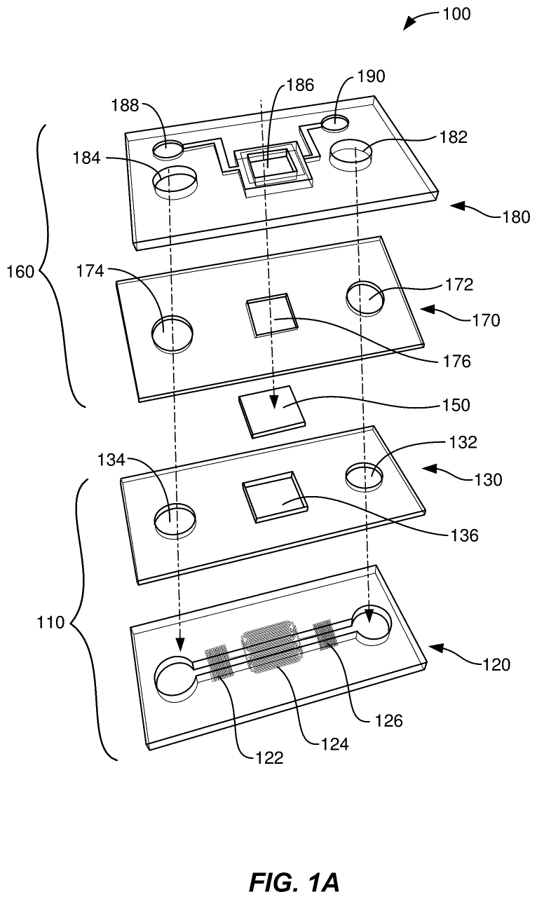

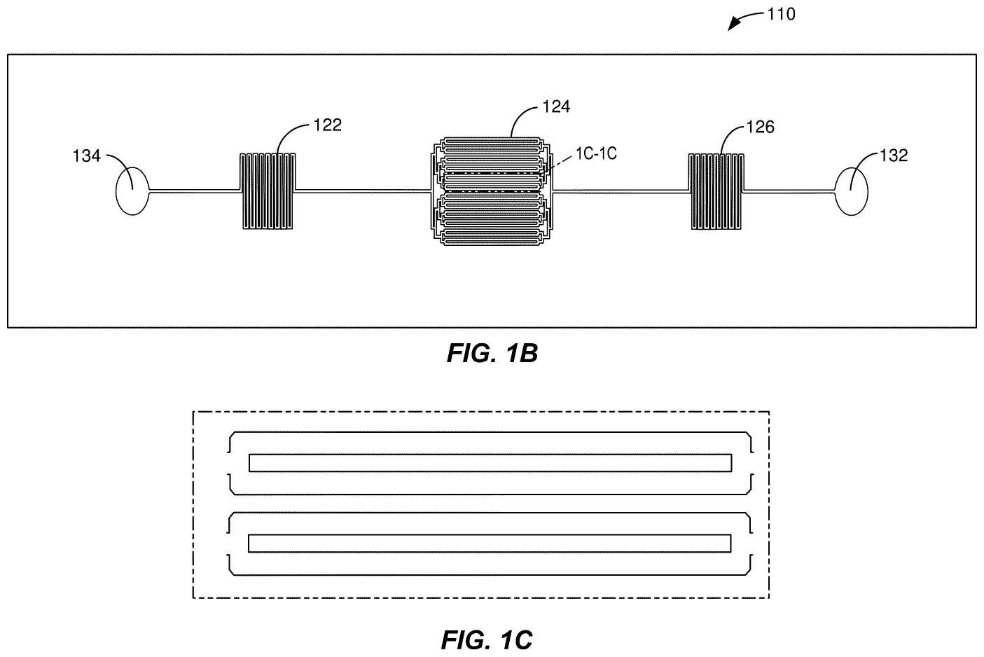

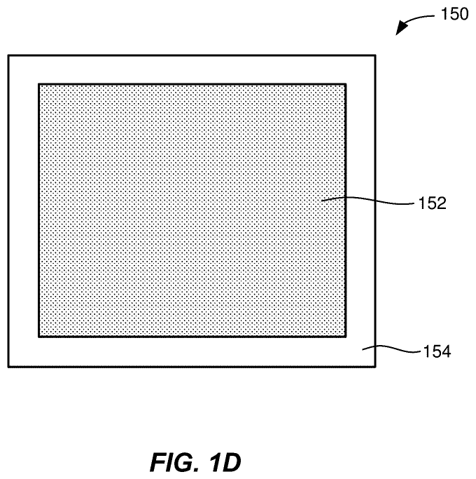

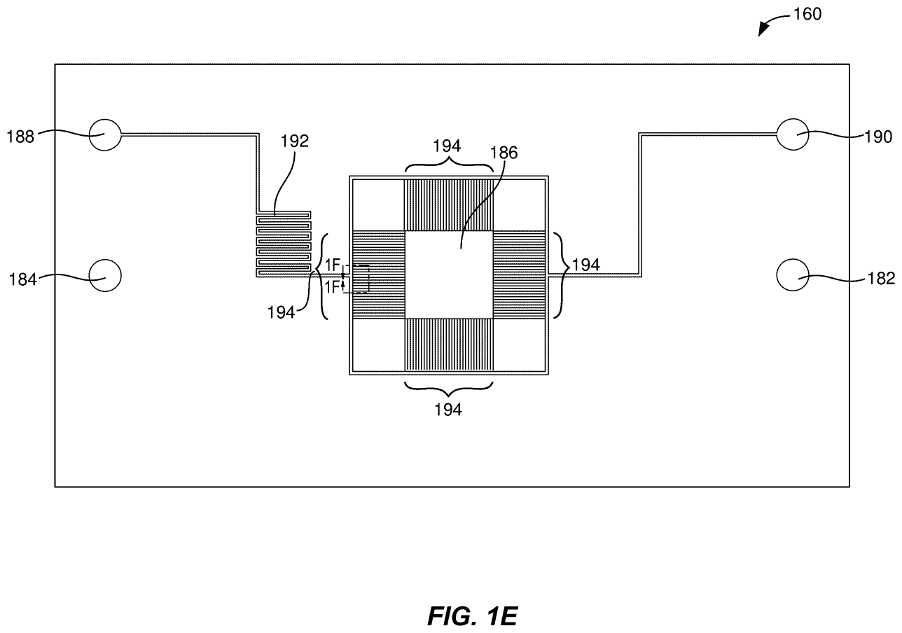

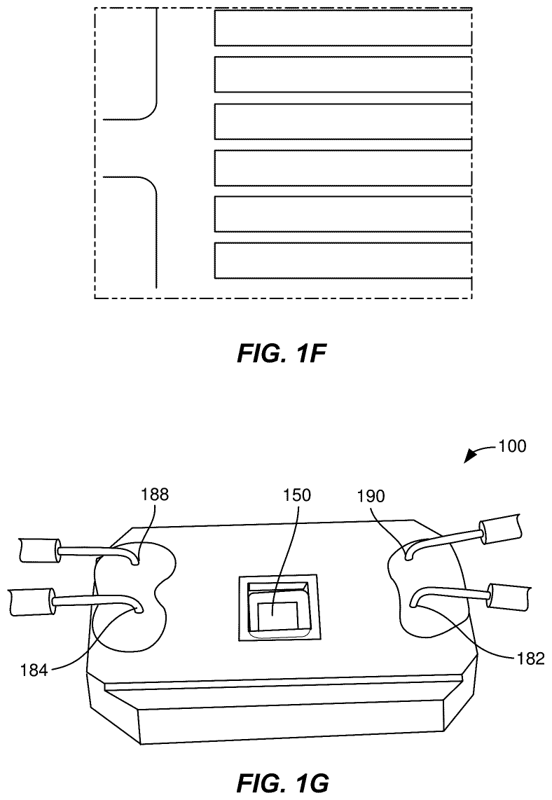

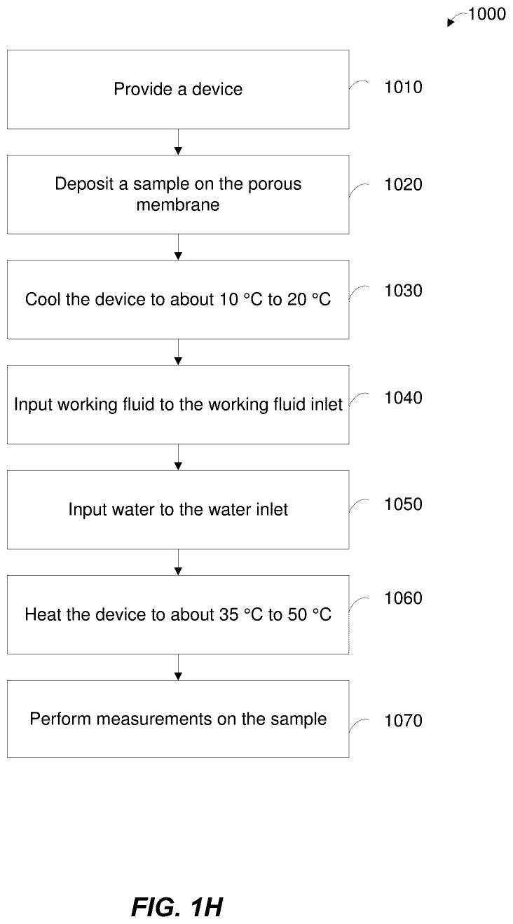

FIGS. 1A-1G show examples of schematic illustrations of a device. Embodiments of the device can be used for Fourier transform infrared (FTIR) spectroscopy, FTIR spectromicroscopy, quantum cascade laser infrared (QCL IR) spectroscopy, and QCL IR spectromicroscopy. FIG. 1A shows an example of an exploded view of the device. As shown in FIG. 1A, a device 100 includes a first assembly 110, a porous membrane 150, and a second assembly 160. In some embodiments, the second assembly 160 is disposed on or over the first assembly 110. In some embodiments, the first assembly is bonded to the second assembly via plasma bonding.

In some embodiments, the first assembly 110 defines a first fluid resistor 122, a fluid distributor 124, and a second fluid resistor 126. In some embodiments, a channel defining the first fluid resistor 122, the fluid distributor 124, and the second fluid resistor 126 has dimensions of about 1 micron to 35 microns by about 1 micron to 35 microns.

In some embodiments, the porous membrane 150 forms a surface of the fluid distributor 124. In some embodiments, the fluid distributor 124 includes a plurality of parallel channels. In some embodiments, the fluid distributor 124 includes about 12 channels to 36 channels, or about 24 channels.

In some embodiments, a fluid resistor comprises a channel that serves to impede or resist the flow of a liquid. In some embodiments, a fluid resistor includes parallel channels that are in fluid communication that run back and forth, e.g., in a repeated “S” or “Z” pattern. In some embodiments, a fluid resistor serves as a passive valve. In some embodiments, a fluid resistor serves to stabilize the flow a liquid downstream from the fluid resistor. A fluid resistor may help to ensure that the fluid flowing to the fluid distributor 124 or the plurality of capillary arrays 194 (described further below) is at a constant flow rate that is not affected by the pump (e.g., a syringe pump, a self-priming diaphragm pump, a peristaltic pump, a piston pump, or a pressure pump, which may become pulsatile and unstable at low flow rates) used to supply a fluid to the device. In some embodiments, a fluid resistor can generate a pressure gradient (i.e., the fluid pressure upstream of a fluid resistor is greater than the fluid pressure downstream of the fluid resistor). In some embodiments, a fluid resistor can further control the flow rate downstream of the fluid resistor. For example, a fluid resistor with a longer length will generate smaller flow rates. In some embodiments, fluid resistors are used to generate symmetrical velocity profiles of the fluid through a cross-section of a channel. Fluid resistors accomplish this by minimizing or eliminating distorted and/or swirling flow of a fluid (e.g., which can generate asymmetrical velocity profiles of the fluid) in the device 100.

In some embodiments, the first assembly 110 comprises a bottom substrate 120 and a top substrate 130. In some embodiments, the bottom substrate 120 is bonded to the top substrate 130 via plasma bonding. In some embodiments, the bottom substrate 120 defines the first fluid resistor 122, the fluid distributor 124, and the second fluid resistor 126. In some embodiments, the top substrate 130 defines a working fluid inlet 134 and a working fluid outlet 132. The top substrate 130 defines a window 136 in which the porous membrane 150 resides. The top substrate 130 also defines a surface of the first fluid resistor 122 and the second fluid resistor 126.

In some embodiments, the first assembly 110 (i.e., both the bottom substrate 120 and the top substrate 130) comprises a polymer from the group polydimethyl siloxane (PDMS), polyamide (PA), polycarbonate (PC), polyester, polyethylene (PE), poly(ethylene terephthalate) (PET), poly(ethylene terephthalate glycol) (PETG), poly(methylmethacrylate) (PMMA), polystyrene (PS), poly(tetrafluoroethylene (PTFE), polyurethane (PU), poly(vinyl chloride) (PVC), and cellulose acetate (C). In some embodiments, the first assembly 110 comprises polydimethylsiloxane (PDMS). In some embodiments, the first assembly 110 is about 1 mm to 3.5 mm thick or about 2.3 mm thick. In some embodiments, the first assembly 110 is thicker than about 3.5 mm. For example, in some embodiments, the bottom substrate 120 of the first assembly 110 is about 1 mm to 3 mm thick or about 2 mm thick. In some embodiments, the top substrate 130 the first assembly 110 is about 50 microns to 500 microns thick or about 300 microns thick.

FIG. 1B shows an example of a top-down view of the first assembly 110, illustrating the first fluid resistor 122, the fluid distributor 124, and the second fluid resistor 126. The first fluid resistor 122, the fluid distributor 124, and the second fluid resistor 126 are connected in series. That is, the first fluid resistor 122 is in fluid communication with the fluid distributor 124. The fluid distributor 124 is in fluid communication with the second fluid resistor 126. FIG. 1C is an enlargement of section line 1C-1C in FIG. 1B, illustrating a number of channels of the fluid distributor 124.

In some embodiments, the porous membrane 150 is a porous membrane selected from the group silicon nitride, silicon dioxide, silicon/silicon dioxide, and graphene. In some embodiments, the porous membrane 150 comprises a silicon nitride porous (SiNₓ) membrane. In some embodiments, a silicon nitride porous membrane is supported by a silicon substrate that defines an opening for the silicon nitride porous membrane. Such a silicon nitride porous membrane is shown in FIG. 1D. FIG. 1D shows an example of an illustration of a porous membrane 150 comprising a silicon nitride porous membrane 152 that is supported by a silicon substrate 154.

In some embodiments, the porous membrane 150 defines a plurality of pores. In some embodiments, each pore of the plurality of pores has a size of about 100 nanometers (nm) to 2 microns, about 100 nm to 900 nm, or about 500 nm. In some embodiments, pores of the plurality of pores in the porous membrane 150 are cylindrical pores. In some embodiments, there is an about 100 nm to 2 micron, about 100 nm to 900 nm, or about 500 nm distance (center-to-center) between pores of the plurality of pores. Pores in the porous membrane 150 should be small enough so that the porous membrane 150 (having an IR reflective metal disposed thereon) reflects IR illumination uniformly with minimal scattering.

In some embodiments, the porous membrane 150 is about 50 nm to 500 microns thick, about 150 microns to 450 microns thick, about 300 nm to 400 microns thick, or about 300 microns thick. In some embodiments, the porous membrane 150 has dimensions of about 3 millimeters (mm) to 5 mm by about 3 mm to 5 mm. In some embodiments, the porous membrane 150 has dimensions of about 3 mm by about 3 mm. In some embodiments, the porous membrane 150 has dimensions of about 5 mm by about 5 mm.

In some embodiments, the fluid distributor 124 serves as a reservoir to provide working fluid to the pores of the porous membrane 150. The working fluid travels (e.g., via imbibition) to the other side of the porous membrane 150 (i.e., the surface of the porous membrane 150 not comprising a surface of the fluid distributor 124 or proximate the fluid distributor 124). In some embodiments, the working fluid comprises a buffer solution (e.g., a solution of sodium acetate (e.g., 1 mM)) or a solution including one or more enzymes.

In some embodiments, the second assembly 160 defines a third fluid resistor 192 (not shown in FIG. 1A) and a plurality of capillary arrays 194 surrounding a window 186 in the second assembly 160 to expose the porous membrane 150. In some embodiments, capillaries of each of the capillary arrays 194 have openings on edges of the window 186 of the second assembly 160. The capillaries are operable to direct moisture across a surface of the porous membrane 150. For example, the plurality of capillary arrays 194 can generate a layer of gas (e.g., air) over a specimen disposed on the porous membrane 150 such that a specified relative humidity proximate the specimen can be maintained.

In some embodiments, the second assembly 160 comprises a bottom substrate 170 and a top substrate 180. In some embodiments, the bottom substrate 170 is bonded to the top substrate 180 via plasma bonding. In some embodiments, the bottom substrate 170 defines a working fluid inlet 174 and a working fluid outlet 172. The bottom substrate 170 also defines a window 176 for the porous membrane 150 and defines a surface of the third fluid resistor 192. In some embodiments, the top substrate 180 defines the third fluid resistor 192 and the plurality of capillary arrays 194 surrounding the window 186. The top substrate 180 further defines a working fluid inlet 184, a working fluid outlet 182, a water inlet 188, and a water outlet 190.

In some embodiments, the second assembly 160 (i.e., both the bottom substrate 170 and the top substrate 180) comprises a polymer from the group polydimethyl siloxane (PDMS), polyamide (PA), polycarbonate (PC), polyester, polyethylene (PE), poly(ethylene terephthalate) (PET), poly(ethylene terephthalate glycol) (PETG), poly(methylmethacrylate) (PMMA), polystyrene (PS), poly(tetrafluoroethylene (PTFE), polyurethane (PU), poly(vinyl chloride) (PVC), cellulose acetate (C), and cyclic olefin copolymer (COC). In some embodiments, the second assembly 160 comprises polydimethylsiloxane (PDMS). In some embodiments, the first assembly 110 and the second assembly 160 each comprise the same polymer. In some embodiments, the second assembly 160 is about 0.4 mm to 1.2 mm thick or about 0.8 mm thick.

FIG. 1E shows an example of a top-down view of the second assembly 160 illustrating the third fluid resistor 192, the plurality of capillary arrays 194 surrounding the window 186, the working fluid inlet 184, the working fluid outlet 182, the water inlet 188, and the water outlet 190. The third fluid resistor 192 and the plurality of capillary arrays 194 are in fluid communication with each other. FIG. 1F is an enlargement of section line 1F-1F in FIG. 1E, illustrating a number of capillaries of one of the capillary arrays.

In some embodiments, a channel defining the third fluid resistor 192 has dimensions of about 1 micron to 35 microns by about 1 micron to 35 microns. In some embodiments, a number of capillaries in each capillary array is about 100 to 1000. In some embodiments, the capillaries of each of the capillary arrays have dimensions of about 1 micron to 35 microns by about 1 micron to 35 microns, or about 20 microns wide by about 10 microns tall. In some embodiments, each of the channels supplying fluid to the respective capillary array (i.e., the channel that is substantially perpendicular to a capillary array that supplies fluid to capillaries of the capillary array) is larger than a capillary of the capillary array (e.g., about 100 microns wide by about 10 microns tall). In some embodiments, the plurality of capillary arrays 194 consists of four capillary arrays. In some embodiments, the window 186 has a square shape, and each of the capillary arrays has openings on one of the sides of the window 186. In some embodiments, the window 186 has dimensions of about 1 mm to 4 mm by about 1 mm to 4 mm.

FIG. 1G shows an example of the assembled device 100. When assembled, the device 100 defines a working fluid inlet 184 in fluid communication with the first fluid resistor 122, a working fluid outlet 182 in fluid communication with the second fluid resistor 126, a water inlet 188 in fluid communication with third fluid resistor 192, and a water outlet 190 in fluid communication with the plurality of capillary arrays 194.

In some embodiments, the device 100 further includes an adhesive membrane (not shown) disposed between the first assembly 110 and the second assembly 160. The adhesive membrane defines a window for the porous membrane. The adhesive membrane serves to join the first assembly 110 and the second assembly 160 (e.g., instead of using plasma bonding). In some embodiments, the adhesive membrane is about 65 microns to 195 microns thick or about 130 microns thick. In some embodiments, the adhesive membrane is a pressure sensitive adhesive membrane.

In some embodiments, a layer of a metal is disposed on a surface of the porous membrane. In some embodiments, the metal reflects infrared light. In some embodiments, the metal is gold. In some embodiments, the metal is about 50 nm to 150 nm thick or about 100 nm thick.

When using embodiments of the device 100 to perform IR spectroscopy or IR spectromicroscopy, a sample is disposed on the porous membrane 150. A pump or pumps (e.g., syringe pump, self-priming diaphragm pump, peristaltic pump, piston pump, or pressure pump) are used to fill the fluid distributor 124 with a working fluid. A heater may also be used to maintain the working fluid at a specified temperature prior to filling the fluid distributor 124. The working fluid is transported from the fluid distributor 124 to the surface of the porous membrane 150 having the sample (e.g., cellulose fibrils) disposed thereon by controlled adsorption and capillary condensation-induced imbibition. The rate of working fluid imbibition through the pores of the porous membrane 150 and the condensation of the working fluid on the surface of the porous membrane can be controlled by a number of parameters, including the size of pores in the porous membrane 150, temperature, and the relative humidity proximate the surface of the porous membrane 150. The rate of working fluid uptake by the sample can be controlled by a number of parameters, including the porosity and surface energy of the sample disposed on the porous membrane 150, temperature, and the relative humidity of the sample.

For the device 100, the relative humidity can be controlled proximate the surface of the porous membrane 150; the rate of water evaporation from areas proximate the surface of the porous membrane having the sample disposed thereon is about the same as the flow rate (i.e., through wicking) of water through the plurality of capillary arrays 194.

In some embodiments, a pump or pumps (e.g., syringe pump, self-priming diaphragm pump, peristaltic pump, piston pump, or pressure pump) are used to introduce water through the water inlet 188 to the plurality of capillary arrays 194. In some embodiments, a syringe without a pump is used to introduce water through the water inlet 188 to the plurality of capillary arrays 194. The water then flows spontaneously through the capillaries of each of plurality of capillary arrays 194 due to the capillary force when water is introduced through the water inlet 188.

In some embodiments, the temperature of a temperature control plate (not shown) on which the device 100 is disposed is set slightly below ambient temperature to prevent evaporation of the water. After the capillary creeping length in the capillaries of each of the plurality of capillary arrays 194 reaches steady state, the temperature of the temperature control plate can be increased to induce evaporation of the water. In some embodiments, the flow rate of water through the plurality of capillary arrays 194 is about 0.1 microliters per minute (μl/min) to 1.5 μl/min. In some embodiments, the humidity proximate the surface of the porous membrane 150 having the sample disposed thereon is slightly below saturation (i.e., about 60%-85% relative humidity). This level of humidity can be maintained for over 12 to 24 hours. This can enable, for example, a real-time FTIR measurement of changing hydrogen bond networks while the sample undergoes enzymatic hydrolysis.

FIG. 1H shows an example of a flow diagram illustrating a method of operating a device. Starting at block 1010 of the method 1000, a device is provided. Any embodiment of the device described herein may be provided.

At block 1020, a sample is deposited on the porous membrane. The sample can be any sample on which measurements are to be performed. In some embodiments, the sample may be prepared on the porous membrane after it is deposited on the porous membrane. For example, the sample may be dried and hydrated to prepare the sample.

At block 1030, the device is cooled to about 10° C. to 20° C. The device can be cooled, for example, by placing it in a refrigerator, with cooling coils that are wrapped around the sides of the device, or by placing the device on a cooling plate.

In some embodiments, operation 1030 is not performed. For example, the environment or room in which measurements on the sample are to be performed may be at a temperature of about 10° C. to 20° C. In such instances, the device would not need to be cooled.

At block 1040, working fluid is input to the working fluid inlet. In some embodiments, working fluid is input to the working fluid inlet until working fluid flows out of the working fluid outlet. This helps to insure that working fluid is present in all of the channels of the flow distributor. In some embodiments, the working fluid input to the working fluid inlet is at about 10° C. to 20° C. The working fluid may need to be cooled to lower it to a temperature of about 10° C. to 20° C.

At block 1050, water is input to the water inlet. In some embodiments, water is input to the water inlet until the each of the channels supplying fluid to the respective capillary array (i.e., the channel that is substantially perpendicular to a capillary array that supplies fluid to capillaries of the capillary array) is full of water. When each of the channels supplying fluid to the respective capillary array is full of water, the water may be drawn down capillaries of the plurality of capillary arrays via capillary force. For example, the capillary force may draw water about ⅔ to ¾ down the length of capillaries of the plurality of capillary arrays.

In some embodiments, the water input to the water inlet is at about 10° C. to 20° C. The water may need to be cooled to lower it to a temperature of about 10° C. to 20° C. With the water and the device at a temperature of about 10° C. to 20° C., the creeping length of water in a capillary of the plurality of capillary arrays is such that it will not flow out of a capillary.

At block 1060, the device is heated to about to slightly above ambient temperature, to about 35° C. to 50° C., or to about 37° C. The temperature to which the device is heated is specified based on the sample on the experiment to be performed. The device may be heated, for example, with a hot plate on which the sample is disposed.

When the device is heated, the working fluid begins to flow (i.e., via imbibition) through pores of the porous membrane. In some embodiments, the working fluid begins to flow through pores of the porous membrane in about 5 seconds to 5 minutes after heating the device to about 35° C. to 50° C. When the working fluid begins to flow through pores of the porous membrane, the working fluid may contact the sample disposed on the porous membrane. If the sample is porous, imbibition of the working fluid will continue through the pores in the sample. If the working fluid reacts with the sample, a reaction may begin.

When the device is heated, moisture also begins to flow from capillaries of the capillary arrays. In some embodiments, a humidity monitor proximate the porous membrane is used to measure the humidity level.

At block 1070, measurements on the sample are performed. For example, FTIR spectroscopy, FTIR spectromicroscopy, quantum cascade laser infrared (QCL IR) spectroscopy, and QCL IR spectromicroscopy may be performed on the sample or on specific regions of the sample.

In some embodiments, once the device is heated to a specified temperature at block 1060, the working fluid will continue to flow through pores of the porous membrane and moisture will continue to flow from capillaries of the capillary arrays. In some embodiments, for short experiments or measurements performed at block 1070 (e.g., up to about 12 hours), no further working fluid and no further water needs to be added to the device after blocks 1040 and 1050, respectively. In some embodiments, for longer experiments or measurements performed at block 1070 (e.g., over about 12 hours), additional working fluid and/or water are added to the device after blocks 1040 and 1050, respectively. That is, additional working fluid and water are added to the device when the measurements on the sample at block 1070 are being performed. The fluid resistor before the plurality of capillary array and the fluid resistor before the flow distributor allow water and working fluid, respectively, to be added to the device at a low flow rate so that measurements on the sample are not interfered with or disrupted.

In some instances, too much working fluid may flow through pores of the porous membrane. In some instances, the working fluid accumulates on the surface of the porous membrane. This may flood the sample, covering the sample with the working fluid, for example. In some embodiments, when too much working fluid begins to flow through pores of the porous membrane, a pump is used to remove working fluid (e.g., through the working fluid outlet) from the flow distributor. For example, data being recorded at block 1070 may show that there is too much working fluid flowing through the porous membrane. A pump would be used to remove working fluid from the flow distributor.

The following examples are intended to be examples of the embodiments disclosed herein, and are not intended to be limiting.

### Example— Device Design

Several design iterations were required to develop an OMM-based device capable of long-term enzymatic reaction and real-time in-situ FTIR analysis of enzymatic hydrolysis of cellulose nanofibrils attached to the membrane. Numerous challenges were encountered in the design process, including: (a) the observation of flooding over the cellulose fibrils from the accumulation of condensates, which obscures the infrared spectral features; (b) the need for reliable moisture dispensing into the reaction chamber for a precise control of the relative humidity to avoid dehydration; (c) increasing sodium acetate concentrations in buffer solution around the fibrils over time; (d) the requirement for a reliable supply of buffer solution from the feeding reservoir below; and (e) the need for multiplexed infrared absorbance detection of enzymatic hydrolysis of cellulose nanofibrils on the surface of the nanomembrane using mid-infrared photons throughout the fingerprint and the OH-hydride regions.

The final device included a number of unique innovations developed in response to these challenges, including: (a) a top-open observation window to allow excess condensate to wick away from the device; (b) a humidity control plate with four high-density capillary arrays to provide a horizontal flow of moisture into the sample reaction chamber; (c) a coupled passive fluid mechanics and thermodynamics mechanism to maintain the relative humidity (rh) in the sample reaction chamber slightly below saturation (˜80%) to minimize evaporation drying, which is accomplished by the coupling of the moisture flow in the high-density capillary arrays and the spontaneous imbibition (nanoporous flow) from the feeding reservoir; (d) at rh=˜80%, the strong surface forces from the water-pore wall attractions lead to condensation near the inlets and induces imbibition, and liquid-like buffer solution migrates through the nanopore membrane and maintains the hydration state of the fibrils; and (e) a FTIR bench and a IR microscope with infrared photons emitted from a broadband mid-infrared source focused by the all-reflective optics infrared microscope with an IR/VIS objective through the observation chamber, the cellulose fibrils, onto the gold-coated nanomembrane inside the AIM (Autonomous Imbibition Microfluidic) device, and are transflected to the IR detector.

The four high-density capillary arrays centered around the humidity control plate were important for reliably providing moisture, in conjunction with the nanopore membrane microfluidic, into the sample reaction chamber to control the humidity environment. Analytic analysis and numerical simulations were performed to identify geometries for the best performance.

### Example— Device Fabrication

One embodiment of the device shown in FIGS. 1A-1G is a stack of three structures with a silicone sample-support plate fixed between two layers of polydimethylsiloxane (PDMS) microchannel structures. A 3 mm×3 mm SiNₓ porous membrane with a thickness of 300 μm and a pore diameter of 500 nm was coated with 100 nm of gold via a sputtering system after being inserted into the opening of the silicone plate. The pore size was small enough that the coated-membrane surface reflected IR illumination uniformly without any noticeable scattering.

PDMS microchannels in the upper humidity control plate and in the bottom buffer reservoir plate were fabricated using standard soft lithography technique. SU-8 3010 (Kayaku Advanced Materials, Westborough, MA) was spin-coated onto a silicon wafer. Sylgard 184 silicone elastomer kit (Dow Chemical Company, Midland, MI) was cast onto the master mold with a 10:1 ratio of elastomer and curing agent followed by curing at 80° C. for 3 hours. The microchannels were 20 μm wide and its thickness was measured to be 10 μm by a profilometer.

The bottom structure was for producing a passive vertical imbibition of buffer solution from the microchannel below through the porous membrane to the sample on the membrane surface. The top structure was a humidity-control microfluidic structure with four microchannel arrays to provide a horizontal flow of moisture that blankets the sample chamber to minimize evaporation or condensation on the porous membrane during the experiment. A vertical through square was carved out using a craft cutter system, followed by plasma bonding to the top microchannel. The plasma treatment formed a sealed two-channel fluid system with individually accessible top and bottom microchannel separated by the porous membranes.

Silicon-based pressure sensitive adhesive tape was used as an intermediate layer to create PDMS-silicone sheet constructs.

### Example— Details of Measurements on Cellulases

Trichoderma reesei Cel7A (TrCel7A), the most studied model cellulases, are cellobiohydrolases (CBHs) that have a catalytic domain (CD) and a cellulose-binding domain (CBD) separated by a highly glycosylated linker. Molecular machine models predict that crystalline cellulose degradation by Cel7A involves the CBD edging itself under a free chain end on the crystalline cellulose surface and feeding it to the CD active site tunnel. The models also predict that forces from these enzyme-substrate interactions could pull cellulose chains from the surface, cleave a cellobiose (Celt) from a reducing end, leaving 13-anomers. These forces also help the enzyme to move on the cellulose chain for processive hydrolysis and Cel2 release. The energy that generates these forces is believed to be derived from the chemical energy of glycosidic bond breakage. This process can be interrogated by infrared absorption spectroscopy. The influence of the Cel7A (TrCel7A) on the spatial changes of microstructure of the highly-crystalline cellulose fibrils isolated from the green algae Cladophora aegagropila 19 at pH 5.0 and two different temperatures (37° C. and 50° C.) was observed over time.

The device can be used to study the enzymatic hydrolysis of cellulose fibril crystalline at low temperature. Experiments were performed at 38° C. and interrogated with SR-FTIR spectromicroscopy measurements. A method of using the device for these experiments includes loading an aliquot of cellulose fibrils suspension into the device and air-drying it overnight. Another aliquot of Cel7A in air-free buffer solution was drop cast onto the air-dried cellulose fibrils. In operations two and three, the ambient temperature (T a) in the device was maintained at −20° C. and regions of interest (ROI) were identified. In operation four, the device was activated and infrared absorption measurements were collected. In operation five, the collected data was analyzed in real time to allow for evaluation of the quality of the experiment and to make immediate changes. Operation five was followed by detailed post-experiment data analysis.

The device was designed to operate with minimal manual intervention. In a typical experiment, the device was could operate for 24 hours, during which at least 30 infrared absorbance marker peaks throughout the mid-infrared region could be recorded. In practice, condensation (and thus interference of the measurement quality) started around 5 hours on each of the experiments, saturating the infrared signal in the hydride-OH spectral region, obscuring chemical bands associated with the inter- and intramolecular hydrogen-bonding interactions.

Triggered by results from the real time analysis, an on-demand stop-flow/draw-down mechanism can be activated to stop/revert flooding over the cellulose fibrils from condensates. In the work described below, these methods were applied to capture spatial and temporal changes in the molecular ordering of cellulose crystalline. Similar to recent reports on methods generating autonomous microfluidics for passive flow, the method is free from moving parts, pumps, valves, and fittings while able to control the highly challenging FTIR measurements.

### Example— Real-Time SR-FTIR Measurements

In the experimental study, the assembled two-channel microfluidic device was mounted on the FTIR microscope stage. The device and its fluid distribution system was connected to the external syringe pump via metallic pins and silicone tubing. The two fluid inputs were controlled by individual syringe pumps, one for injecting deionized (DI) water into the top microchannel; the other for injecting acetate buffer solution into the bottom reservoir microchannel. Before the experiment, 60 μL of algal cellulose (0.025 mg/mL) was deposited and air-dried onto the membrane. 20 μL of cellulase TrCel7A (0.5 μmole/g) was introduced and allowed to infiltrate through the cellulose at room temperature (˜20° C.); dehydration followed immediately to prevent initiation of enzymatic activity. This dehydration process took 40 minutes to complete at room temperature.

To initiate enzymatic hydrolysis, the temperature of the FTIR microscope stage was raised to 37° C. Sodium acetate buffer solution (1 mM) was injected at a flow rate of 0.3 μL/min into the fluid distribution channel connected to the bottom microchannel. The acetate solution rose vertically by capillary forces through the SiNₓ pores to provide moisture to the cellulose on the SiNₓ membrane surface. Simultaneously, DI water was injected at a flow rate of 0.2 μL/min into the fluid distribution system connected to the capillary arrays in the humidity control chamber to maintain a relative humidity of −60% around observation window at 37° C. Similarly, DI water in the fluid distribution channel was pulled into the arrays of microchannels due to capillary force, followed by evaporation into the observation window. At 37° C., the system maintained a relative humidity of −60%. The evolution of the distribution of humidity and temperature were confirmed by Computational Fluid Dynamics (CFD) simulations.

Continuous real-time measurements of cellulose dehydration or enzymatic hydrolysis were performed using synchrotron-radiation infrared illumination at Beamline 1.4.3 at the Advanced Light Source at Lawrence Berkeley National Laboratory. In the dehydration experiment, Synchrotron FTIR (sFTIR) measurements were performed on an IR microscope with a 32×, 0.65 numerical aperture objective with a FTIR spectrometer using a KBr beamsplitter and a single pixel (HgCdTe) detector. sFTIR spectra were collected in reflection mode between 650 cm−1 and 4000 cm⁻¹ at 4 cm⁻¹ spectral resolution and recorded with 32 co-added scans at a 5 μm step size. sFTIR imaging of sample areas were performed using raster scanning method.

Using the microfluidic device, small changes to cellulose molecular ordering due to hydration were observed. Further, continuous, live imaging in a 20×20 μm area of cellulose was performed, collecting IR spectral data in using an 8×8 grid within the observed area every 20 minutes over 14 hours of reaction. The resulting IR spectra provided insight into spatially resolved hydrolysis kinetics and changes in molecular ordering of cellulose over time due to enzyme action.

Time-elapsed continuous mapping of sample areas were conducted using a control system. The control system controlled the movement of the microscope stage, spectra collection of the FTIR spectrometer, and data storage.

### Example— sFTIR Spectra of Cellulose

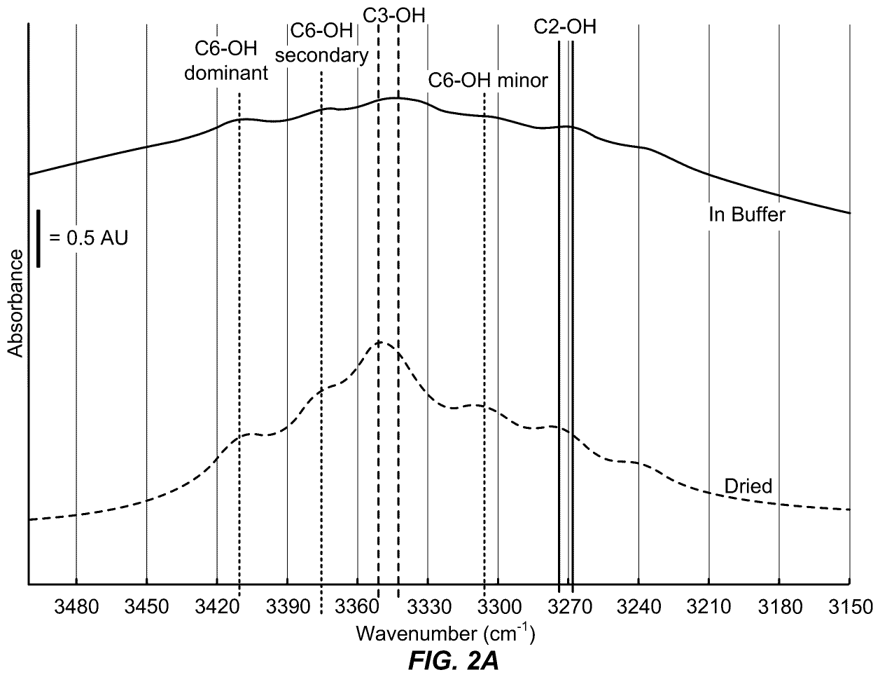

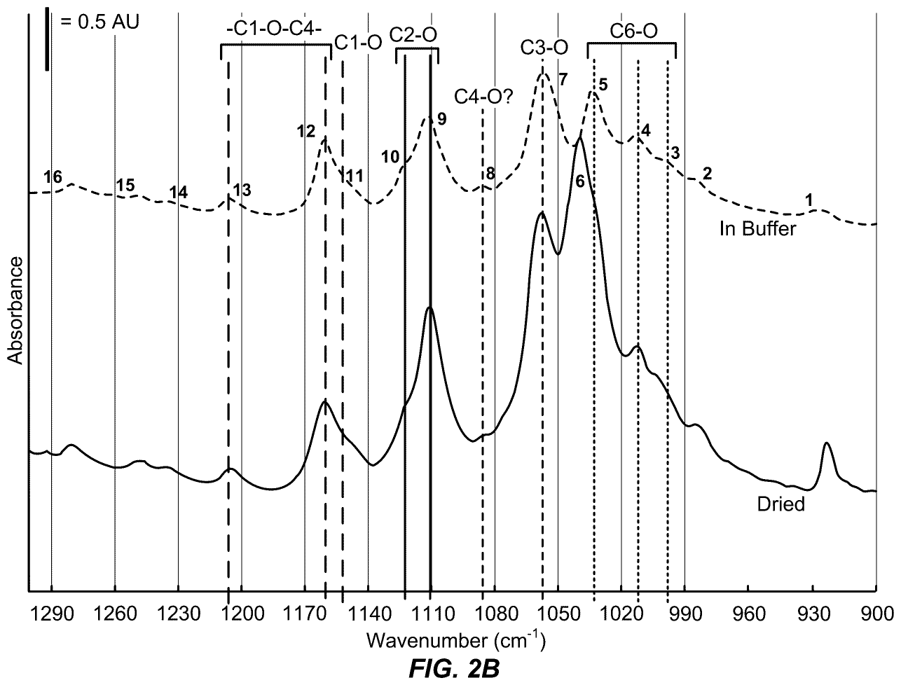

The sFTIR spectra of cellulose feature signature absorbance peaks in the hydrogen-bonding region (˜3200-3400 cm⁻¹) and in the fingerprint region (900-1300 cm⁻¹) are shown in FIGS. 2A and 2B. Consistent with previous reports, the fingerprint region of the cellulose sFTIR spectrum shows strong absorbance peaks due to C—O bond vibrations at the 2nd, 3rd and 6^{th} carbons of the glucose residues of cellulose (C2, C3, and C6, respectively) centered at −1111 cm⁻¹ (peak 9), 1060 cm⁻¹ (peak 7), and 1034 cm⁻¹ (peak 5), respectively. The asymmetric and symmetric stretching of the —C—O—C ether bond of the glycosidic bond in cellulose absorb maximally near 1160 cm⁻¹ (peak 12) and 1206 cm⁻¹ (peak 13), respectively. The asymmetric stretching peak of the glycosidic bond at −1160 cm⁻¹ is dominant and commonly used as a diagnostic marker for cellulose. Additionally, a shoulder peak centered at 1153 cm⁻¹ (peak 11) attributed to C—O stretching of the anomeric carbon can be seen in the spectra. The corresponding identification of the C—O stretch at the 4^{th} carbon (i.e., non-reducing ends of cellulose, FIG. 2A) is not commonly identified in the literature. A minor peak centered at −1086 cm⁻¹ (peak 8) is a possible candidate for C4—O vibration. The small peak size comparable to the shoulder peak at 1153 cm⁻¹ of the reducing-end, and the location between the C3—O and C2—O peaks, where C2-OH, C3—OH, and C4—OH are secondary alcohols, lend support to this hypothetical assignment.

Corresponding to the C—O stretches are C—OH vibration peaks that occur in the 3200-3400 cm−1 range (FIG. 2B). Most notably—OH vibration peaks at C2—OH, C3—OH and three conformations of C6OH in cellulose are identifiable at −3270 cm⁻¹, ˜3350 cm⁻¹, and 3305 cm⁻¹, 3374 cm⁻¹, and 3410 cm⁻¹, respectively. The hydroxymethyl group at C6 along the cellulose backbone can rotate about the C5-C6 bond in each glucose residue and occupy three dominant rotameric forms. The ratio of the three rotamers at the cellulose surfaces strongly depends on the nature of the solvent, while the rotameric form facilitating intermolecular hydrogen bonds between C6—OH and C2—OH dominate within the fibril structures. The dominant rotamer of C6-OH absorbs maximally at 3410 cm⁻¹ with the corresponding C6—O stretch absorbing at −1034 cm⁻¹. The less dominant rotamers of C6—OH absorb at 3305 cm⁻¹ and 3374 cm⁻¹, with corresponding C6—O peaks at −1013 cm⁻¹ and 997 cm⁻¹.

### Example—Hydration Increases Surface Ordering of Cellulose

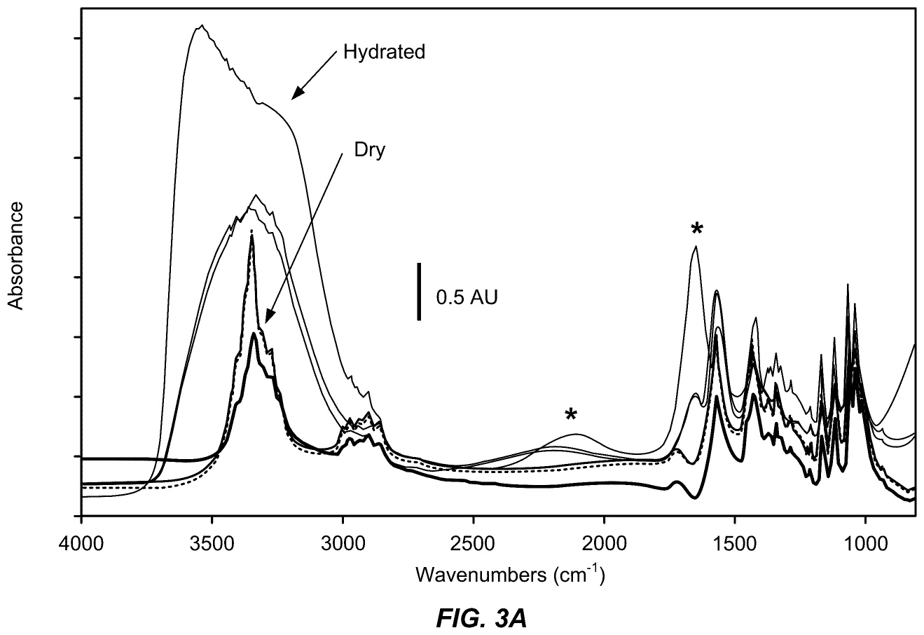

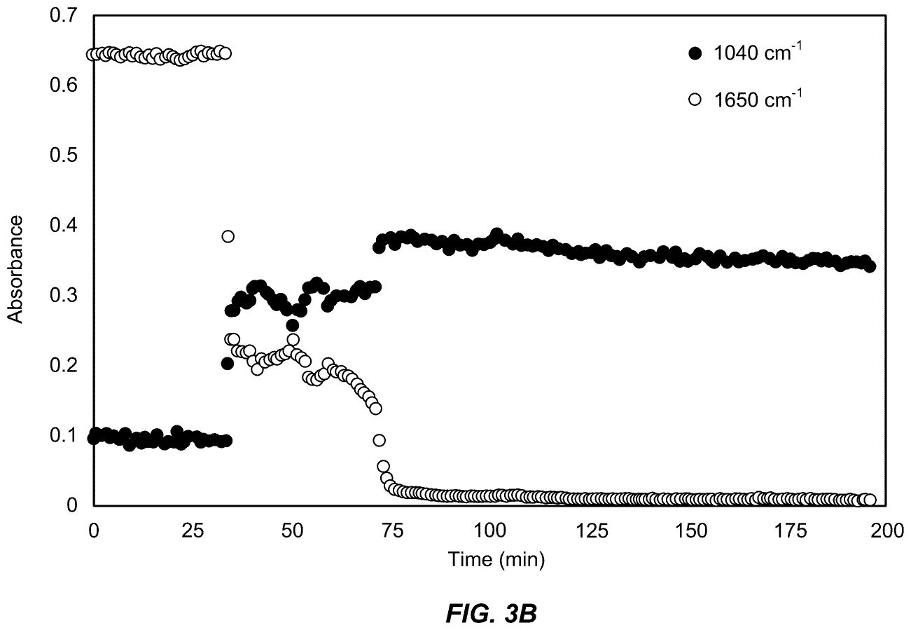

An experiment was conducted in the humidity-controlled microfluidics device where time resolved spectra were collected over 200 minutes during which initially hydrated cellulose was dried (FIG. 3A). In this experiment, cellulose remained fully hydrated for about the first 30 minutes, after which a sharp drop in the absorption amplitude of the 1650 cm⁻¹ peak could be seen (FIG. 3B). A second sharp drop in the amplitude of the 1650 cm⁻¹ peak to zero at about 75 minutes further indicated full removal of bound moisture (i.e., fully dried cellulose). Interestingly, the timing of the decrease in the 1650 cm⁻¹ signal corresponded to step increases in the amplitude of the peak centered at 1040 cm⁻¹ wavenumbers. Absorption at 1040 cm⁻¹ in the fingerprint region is assigned to C—O vibrations of amorphous cellulose. Cellulose exhibited lower crystallinity indices (CI) in a dry state than in a hydrated state. In the experiments, the 1040 cm⁻¹ peak was only apparent in dry cellulose samples (FIGS. 2A, 2B, 3A, and 3B). Moreover, some shifts in the peak centers of the cellulose IR spectra occurred upon hydration, most notably in the hydrogen-bonding region (3200-3500 cm⁻¹) (FIG. 2A). When hydrated in buffer, the C3—OH and C2—OH peak centers shifted to lower wavenumbers indicating weakening of the —OH bond due to stronger hydrogen bonding (3350 cm⁻¹ to 3341 cm⁻¹, and −3275 cm⁻¹ to 3270 cm⁻¹, respectively) (FIG. 2A).

Both C3—OH and C2—OH participate in intra- and intermolecular hydrogen bonds within the cellulose structure. Wavenumber shifts of peaks in the fingerprint region of cellulose (900-1300 cm⁻¹, FIG. 2B) were also observed but to lesser extents of 1-2 wavenumber increases, generally indicating a stiffening of the cellulose structure in water. Taken together, the lack of a C—O vibration peak of amorphous cellulose and peak shifts to higher wavenumbers suggest that association with moisture results in increasing ordering of cellulose.

### Example—Time- and Spatially-Resolved FTIR Spectra of Cellulose Undergoing Enzyme Hydrolysis

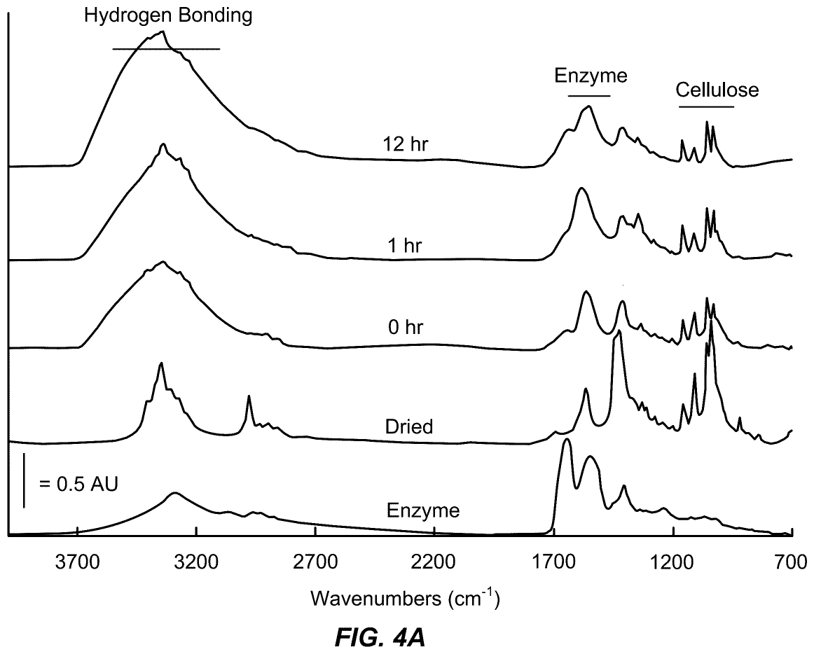

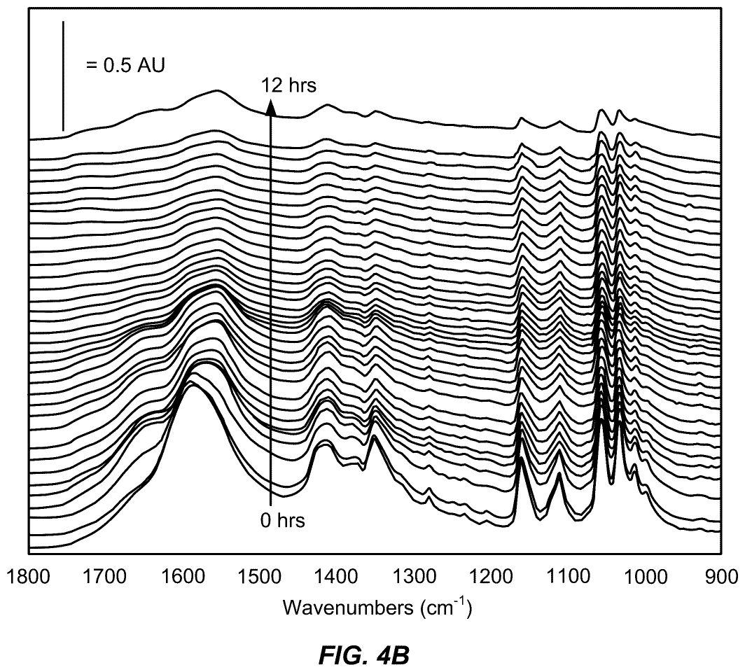

The cellulose dehydration experiment demonstrated the utility of the humidity-controlled microfluidics platform for documenting time-resolved changes in cellulose structure by sFTIR. Similarly, IR spectral evolution of cellulose undergoing hydrolysis by a purified Cel7A in buffer was recorded (FIGS. 4A and 4B).

In general, in the presence of buffer, the signal in the 3000-3700 cm⁻¹ wavenumbers region was dominated absorption by bulk water (FIG. 4A). Additionally, peaks corresponding to sodium acetate could be seen between 1400-1450 cm⁻¹ and 1570 cm⁻¹ in the spectra containing buffer. In samples with Cel7A, amide I and amide II peaks of the enzyme centered around 1650 cm⁻¹ and 1550 cm⁻¹, respectively, were seen in all but the dried cellulose spectrum where enzyme was not added (FIG. 4A). Note that the 1650 cm⁻¹ absorption peak also corresponds to adsorbed water as discussed above. To deconvolute the effects of buffer and free enzyme, the spectra of cellulose undergoing enzymatic hydrolysis were background corrected by subtracting the spectra of free enzyme in buffer (FIG. 4B).

The sFTIR spectra of Cel7A hydrolysis of cellulose were recorded every 20 minutes over a 14-hour period (FIG. 4B). The cellulose peaks in the fingerprint region, particularly centered around 1160 cm⁻¹ (glycosidic bond), 1112 cm⁻¹ (C2—O), 1060 cm⁻¹ (C3—O), and 1034 cm⁻¹ (C6—O) are clearly distinguishable in the background-corrected, time-resolved spectra. Signal in the 1500-1700 cm⁻¹ region remaining after correction are thus due to adsorbed enzyme and moisture. Overall, the characteristic cellulose peaks decreased in amplitude over the course of hydrolysis, indicating a general decrease in their abundance; i.e., cellulose removal due to enzymatic hydrolysis.

### Example—Cellulose Removal Due to Enzymatic Hydrolysis

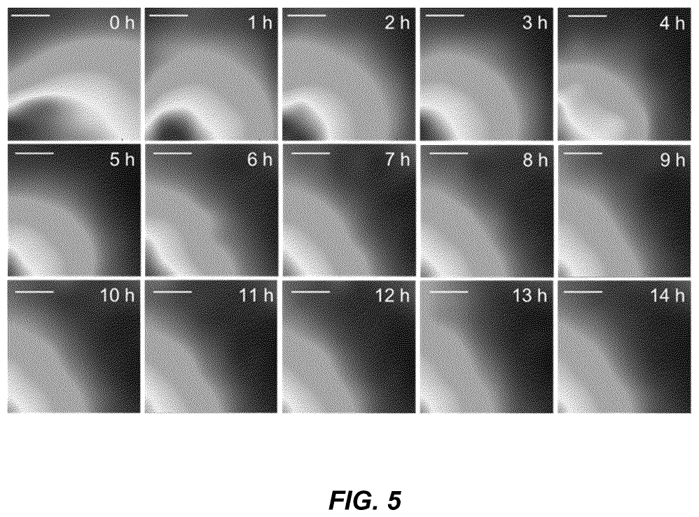

While the time-resolved spectra in FIG. 4B showed overall depletion of the cellulose due to hydrolysis, the distribution of cellulose and the rates of depletion were heterogeneous in the imaged region. The lower half of the imaged region had a thicker deposition of cellulose, with more thinly distributed cellulose fibrils radiating out into the upper half. This uneven distribution of cellulose in the imaged region can be visualized as a heat map of the amplitude of the glycosidic bond peak of cellulose at −1160 cm⁻¹ wavenumbers (0 h in FIG. 5). In general, high intensities corresponding to high peak amplitudes mapped to areas with the thicker layer of cellulose, and decreased intensities radiated outwards where cellulose appeared more thinly distributed. Over time, there was an overall decrease in the ˜1160 cm⁻¹ peak amplitude, with an apparent shrinking of the cellulose ‘particle’. By 4 hours, the signal from the ˜1160 cm⁻¹ peak was only in the bottom left quadrant of the imaged region.

Indeed, the amplitude of the ˜1160 cm⁻¹ peak decreased faster near the particle edges, in accordance with previous observations of a surface erosion mechanism. Within 4 hours, signal from the ˜1160 cm⁻¹ peak was confined in the bottom left quadrant of the imaged region. The rates of amplitude decrease at each point within the imaged region reflect trends reminiscent of typical cellulose hydrolysis kinetics with an initial rapid rate of depletion. In regions with thicker cellulose, the rates of depletion leveled off and remained relatively constant. The cellulose used in this study was a highly recalcitrant algal cellulose, previously shown to achieve low extents of conversion in bulk hydrolysis reactions. In reactions with Cel7A, the hydrolysis rates of algal cellulose plateau despite excess cellulose remaining because of a depletion of productive binding sites.

### Example—Enzymatic Hydrolysis Impact Local Molecular Ordering of Cellulose

In addition to peak intensity, the peak locations of characteristic cellulose peaks throughout hydrolysis were tracked, as shifts in the peak location can provide information about the changes in length and associated strength of chemical bonds. A shift towards higher wavenumbers is indicative of a stiffer bond, resonating at higher energies, while a shift towards lower wavenumbers suggests weakening of the bond. Over the course of hydrolysis, peaks associated with cellulose, including the glycosidic bond vibration at −1160 cm⁻¹, exhibited average increases in wavenumber over the entire imaged region.

Closer examination, however, revealed location-dependent wavenumber shifts. Over the course of 14 hours in the observed region of cellulose, the 1160 cm⁻¹ glycosidic bond peak shifted to higher wavenumbers at some locations, but also shifted to decreasing wavenumbers or did not significantly shift at some locations. An increasing wavenumber shift of the glycosidic bond peak can either indicate stiffening of the cellulose backbone, or that cellulose with less stiff glycosidic bonds were removed during enzymatic hydrolysis. An examination of the ˜1034 cm⁻¹ C6—O and −1060 cm⁻¹ C3—O peaks similarly reveal an overall shift towards higher wavenumbers in the imaged region, resolved into localized increased and decreased shifts. The overlay of trends in average wavenumber shift of the ˜1034 cm⁻¹ C6—O, ˜1060 cm⁻¹ C3—O and 1160 cm⁻¹ glycosidic bond peaks show that all three peaks shifted to decreasing wavenumbers in the region with the thickest deposition of cellulose, but shifted to increasing wavenumbers where there appeared to be less cellulose.

The −1034 cm⁻¹ peak in the cellulose FTIR spectra is due to the vibration of the C6—O bond. In the cellulose fibril, the oxygen of C6—O is involved in O2H—O6 hydrogen bonding within the cellulose molecule and O6—HO2 between cellulose molecules. These intra- and intermolecular hydrogen bonds can physically constrain the C6—O bond. Decrystallization of a cellulose molecule from the fibril structure disrupts the O6—HO2 intermolecular hydrogen bonds, which alleviates constraint of the bond that would manifest in the IR spectra as a decreasing wavenumber shift of the C6—O vibration peak. Thus, regions where the ˜1034 cm⁻¹ peak shifts towards lower wavenumbers over the course of hydrolysis suggest active decrystallization of cellulose. The complexation of Cel7A with cellulose necessitates decrystallization of a molecule from the fibril surface. Thus, these may be regions where Cel7A are actively complexing to and possibly hydrolyzing cellulose.

The −1060 cm⁻¹ peak arises from the vibration of the C3—O bond, which is involved in the 03—HO6 intermolecular hydrogen bond. Thus, decrystallization of a cellulose molecule from the fibril will also lessen the physical constraint on the C3—O bond, allowing the bond to resonate at lower wavenumbers. Similar to the ˜1034 cm⁻¹ peak, decreasing shifts in the ˜1060 cm⁻¹ peak suggests regions with active decrystallization of cellulose over the course of hydrolysis.

In the general region where all three ˜1034 cm⁻¹, ˜1060 cm⁻¹, and −1160 cm⁻¹ peaks shift to lower wavenumbers, the amplitude of the 1160 cm⁻¹ peak also show a decreasing trend. As noted, the brightfield image shows this to be a region with a thicker deposition of cellulose. Taken together, the trends in peak shift and amplitude change suggest active cellulase activity in this region (i.e. decrystallization by complexation, and hydrolysis). Outside of this region, this triad of −1034 cm⁻¹, ˜1060 cm⁻¹, and −1160 cm⁻¹ peaks tend to shift to increasing wavenumbers. These appear to be regions where the 1160 cm⁻¹ peak amplitude exhibit only small changes or plateau during the observed time. Increasing wavenumber shifts suggests a removal of less-ordered cellulose due to hydrolysis.

### Example—Discussion

Spatial heterogeneity in the hydrolysis of cellulose by cellulases is posited to contribute strongly to the overall recalcitrance of cellulose, but is challenging to characterize. IR spectromicroscopy enables highly resolved spatial mapping of material composition by bond vibrational modes. As described above, IR spectromicroscopy enabled spatial mapping of changes to cellulose composition and molecular order imparted by enzyme action. Moreover, the use of an open-channel microfluidics system to control temperature, moisture, and humidity levels facilitated data collection over 14 hours in an aqueous buffered reaction. Moisture has been a major impediment to IR spectromicroscopy because of strong infrared absorption by water. While water is a critical component of most biological reactions, including cellulose hydrolysis, water has also been implicated in imparting changes to molecular ordering of cellulose crystalline fibrils. The extent of intrafibril hydrogen bonding within cellulose fibrils contribute to the crystalline morphology and crystallinity of cellulose. Water-cellulose and cellulose-cellulose hydrogen bonding at the surface of cellulose cannot only affect ordering of surface chains, but disorder could propagate into the cellulose fibrils structure.

The precise role of ‘crystallinity’ in the resistance of cellulose to hydrolysis has been difficult to elucidate. Most often, the total crystallinity index (TCI), a bulk measure of the fraction of ordered cellulose in a given sample, is used to query the relationship between crystallinity and cellulose digestibility. However, cellulose TCI, including those estimated from peak ratios in the FTIR spectra of cellulose, remains an ambiguous measure of cellulose digestibility. This is not to say that cellulose crystallinity or molecular ordering of cellulose molecules, particularly at the water-fibril interface, is irrelevant to enzymatic hydrolysis. Cellulase hydrolysis of cellulose is an interfacial reaction where cellulases must decrystallize and complex to isolated cellulose molecules at the surface of the fibrils. Alternatively, cellulases must locate a decrystallized (or amorphous) cellulose molecule at the surface of the fibrils to complex with. Thus, a reasonable hypothesis is that localized surface disorder in molecular arrangement offers more productive binding sites. In other words, a measure of cellulose ordering with spatial resolution is needed.

As described above, spatially resolved changes in localized molecular ordering of cellulose was assessed from the ‘blue shift’ (increasing wavenumber), ‘red shift’ (decreasing wavenumber), or intensity change of key peaks of cellulose in the IR fingerprint region. The absorption peaks of the glycosidic bond, C3—O and C6—O bonds were used to gauge changes in chain stiffness and molecular ordering. Glycosidic bonds along the backbone of cellulose are braced on either side by the O5-H03 and O2H—O6 intramolecular hydrogen bonding. The bracing imparts rigidity to the cellulose molecule such that cellooligosaccharides decrease in solubility with increasing degrees of polymerization (DP), and chains with DP≥6 are insoluble in water. Moreover, the O2H—O6 hydrogen bond, occurring on alternating sides of the glycosidic bonds along the molecule, is suggested to strain the molecule and cause the right-handed twist observed in cellulose fibrils. Previous studies have reported physical manifestation of the release of stress within the fibrils during enzymatic hydrolysis as spontaneous untwisting and kinking of fibrils. The study described above demonstrated spatial heterogeneity in molecular ordering trends during hydrolysis, where regions with rapid depletion of cellulose generally increased in molecular ordering while regions with on-going or incomplete cellulose depletion had decreased molecular ordering. These trends suggest that less-ordered cellulose is depleted, leaving more ordered cellulose; and active hydrolysis decreases cellulose molecular ordering, possibly because of active complexation of enzymes at the cellulose surface. Earlier studies correlating IR spectral changes with enzymatic hydrolysis trends have not reported shifts in peak centers because dried bulk samples were measured. This study clearly demonstrates localized changes in molecular ordering that could be averaged out over the entire sample or altered when dried.

The supposition of surface disordering of cellulose in regions of active enzymatic hydrolysis has interesting implications for understanding cellulose recalcitrance. An unanswered question from this study is whether cellulase action creates disorder at the cellulose surface, or do disordered cellulose surfaces invite active cellulase complexation. A more detailed study with higher spatial resolution may be necessary to address this question, to help inform future efforts in enzyme engineering to improve enzyme's ability to decrystallize and complex to cellulose, and biomass pretreatment strategies targeting desired cellulose surface properties.

### CONCLUSION

In situ, spatial mapping of the infrared spectra of cellulose during enzymatic hydrolysis in buffer was successfully achieved using an open-channel humidity controlled microfluidics device. The spatial heterogeneity in hydrolysis rates and changes in molecular ordering of cellulose over the course of hydrolysis by Cel7A was successfully documented. Hydrolysis by Cel7A preferentially removes less-ordered cellulose, leaving behind more ordered cellulose. Complexation of Cel7A to cellulose required decrystallizing cellulose at the surface, or regions of disorder where Cella can find productive binding sites to complex to.

In the foregoing specification, the invention has been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

What is claimed is:

1. A device comprising:
a first assembly, the first assembly defining a first fluid resistor, a fluid distributor, and a second fluid resistor, the first fluid resistor, the fluid distributor, and the second fluid resistor being connected in series;
a porous membrane overlying the fluid distributor, the porous membrane forming a surface of the fluid distributor; and
a second assembly, the second assembly disposed on the first assembly, the second assembly defining a third fluid resistor and a plurality of capillary arrays surrounding a window in the second assembly that exposes the porous membrane, the third fluid resistor and the plurality of capillary arrays being in fluid communication;
the device further defining a working fluid inlet in fluid communication with the first fluid resistor, a working fluid outlet in fluid communication with the second fluid resistor, a water inlet in fluid communication with third fluid resistor, and a water outlet in fluid communication with the plurality of capillary arrays, and capillaries of each of the capillary arrays having openings on edges of the window of the second assembly and being operable to direct moisture across a surface of the porous membrane.

2. The device of claim 1, wherein the porous membrane is a porous membrane selected from the group silicon nitride, silicon dioxide, silicon/silicon dioxide, and graphene.

3. The device of claim 1, wherein the porous membrane is a silicon nitride porous membrane.

4. The device of claim 1, wherein the porous membrane is about 50 nanometers to 500 microns thick.

5. The device of claim 1, wherein the porous membrane has dimensions of about 3 millimeters to 5 millimeters by about 3 millimeters to 5 millimeters.

6. The device of claim 1, wherein the porous membrane defines a plurality of pores, and wherein each pore of the plurality of pores has a size of about 100 nanometers to 2 microns.

7. The device of claim 1, wherein a layer of a metal is disposed on the surface of the porous membrane.

8. The device of claim 1, wherein a number of capillaries in each capillary array is about 100 to 1000.

9. The device of claim 1, wherein the plurality of capillary arrays consists of four capillary arrays, wherein the window has a square shape, and wherein each of the capillary arrays has openings on one of the sides of the window.

10. The device of claim 1, wherein the window has a square shape, and wherein the window has dimensions of about 1 millimeter to 4 millimeters by about 1 millimeter to 4 millimeters.

11. The device of claim 1, wherein the capillaries of each of the capillary arrays have dimensions of about 1 micron to 35 microns by about 1 micron to 35 microns.

12. The device of claim 1, wherein a channel defining the third fluid resistor has dimensions of about 1 micron to 35 microns by about 1 micron to 35 microns.

13. The device of claim 1, wherein channels defining the first fluid resistor, the fluid distributor, and the second fluid resistor have dimensions of about 1 micron to 35 microns by about 1 micron to 35 microns.

14. The device of claim 1, wherein the first assembly and the second assembly each comprise a polymer from the group polydimethyl siloxane (PDMS), polyamide (PA), polycarbonate (PC), polyester, polyethylene (PE), poly(ethylene terephthalate) (PET), poly(ethylene terephthalate glycol) (PETG), poly(methylmethacrylate) (PMMA), polystyrene (PS), poly(tetrafluoroethylene (PTFE), polyurethane (PU), poly(vinyl chloride) (PVC), cellulose acetate (C), and cyclic olefin copolymer (COC).

15. The device of claim 1, wherein the first assembly is about 1 millimeter to 3.5 millimeters thick.

16. The device of claim 1, wherein the second assembly is about 0.4 millimeters to 1.2 millimeters thick.

17. The device of claim 1, further comprising:
an adhesive membrane disposed between the first assembly and the second assembly.

18. The device of claim 1, wherein the first assembly is bonded to the second assembly via plasma bonding.

19. A method comprising:
providing a device, the device comprising:
a first assembly, the first assembly defining a first fluid resistor, a fluid distributor, and a second fluid resistor, the first fluid resistor, the fluid distributor, and the second fluid resistor being connected in series;
a porous membrane overlying the fluid distributor, the porous membrane forming a surface of the fluid distributor; and
a second assembly, the second assembly disposed on the first assembly, the second assembly defining a third fluid resistor and a plurality of capillary arrays surrounding a window in the second assembly that exposes the porous membrane, the third fluid resistor and the plurality of capillary arrays being in fluid communication;
the device further defining a working fluid inlet in fluid communication with the first fluid resistor, a working fluid outlet in fluid communication with the second fluid resistor, a water inlet in fluid communication with third fluid resistor, and a water outlet in fluid communication with the plurality of capillary arrays, and capillaries of each of the capillary arrays having openings on edges of the window of the second assembly and being operable to direct moisture across a surface of the porous membrane;
depositing a sample on the porous membrane;
cooling the device to about 10° C. to 20° C.;
inputting working fluid to the working fluid inlet;
inputting water to the water inlet;
heating the device to about 35° C. to 50° C.; and
performing measurements on the sample.
